(19) United States
(12) Patent Application Publication
(10) Pub. No.: US 2009/0240219 A1
(43) Pub. Date: Sep. 24, 2009

(54) One-piece colostomy pouch

(76) Inventor: Stanley Barcroft, Havant (GB)

Correspondence Address: MR. STANLEY BARCROFT, 1 ELDER ROAD, HAVANT, HAMPSHIRE, P09 2UN, GB

(21) Appl. No.: 12/218,960

(22) Filed: Jul. 21, 2008

(30) Foreign Application Priority Data
Mar. 19, 2008 (GB) GB 0805056.9

(51) Int. Cl. A61F 5/44

(52) U.S. Cl. 604/332

(57) ABSTRACT

Convexity of the abdomen, as with a parastomal hernia, can cause problems in fitting a pouch by the standard procedure of using the fingers of both hands to apply pressure over and around the adhesive wafer. The mismatch of the surfaces leads to the formation of folds and wrinkles which can cause leakage in service.

This invention introduces a handle fixed to the front of the pouch which facilitates placing the wafer over the stoma, followed by compression of the stoma and hernia, during one simple push by the palm of the hand. Sealing takes place radially outwards from the stoma and results in two flat surfaces bonded together. Simultaneously the warmth of the hand assists the bonding process. On release of the pressure, wrinkling inevitably occurs but this is shallow and wavelike, the skin conforms neatly to it, and no leakage subsequently takes place.

A typical one-piece colostomy pouch embodies an adhesive wafer which fits closely around the stoma where it emerges from the abdomen. The standard procedure for fixing a pouch in place is to fold the wafer backwards and press it firmly against the abdomen, smoothing it upwards with the fingers of both hands. This can be a difficult and haphazard process.

Problems arise when the abdomen is rounded, especially when a parastomal hernia is present. The mismatch between the surfaces gives rise to folds and wrinkles which can cause subsequent leakage.

The invention overcomes this problem and also provides other advantages when compared with the standard product and its recommended fitting procedure.

The improved pouch embodies a handle fixed to the front cover of the pouch which facilitates the holding and carrying of the pouch on the opened palm of one hand. In one simple pushing movement the pouch is first located over the stoma followed by a progressive compression of the rounded surface of the abdomen with contact commencing at the stoma and automatically spreading radially outwards until the periphery of the wafer is reached. This results in two flat surfaces bonded together. If the pressure is maintained for about one minute, the warmth of the hand softens the adhesive and improves the sealing process.

On removal of the hand some wrinkling inevitably occurs, but this is shallow and wavelike, to which the skin conforms neatly and there is no subsequent leakage.

Details of the present invention, will now be described, purely by way of example, with reference to the accompanying drawings in which:

FIG. 1 is a front view of a typical one-piece colostomy pouch.

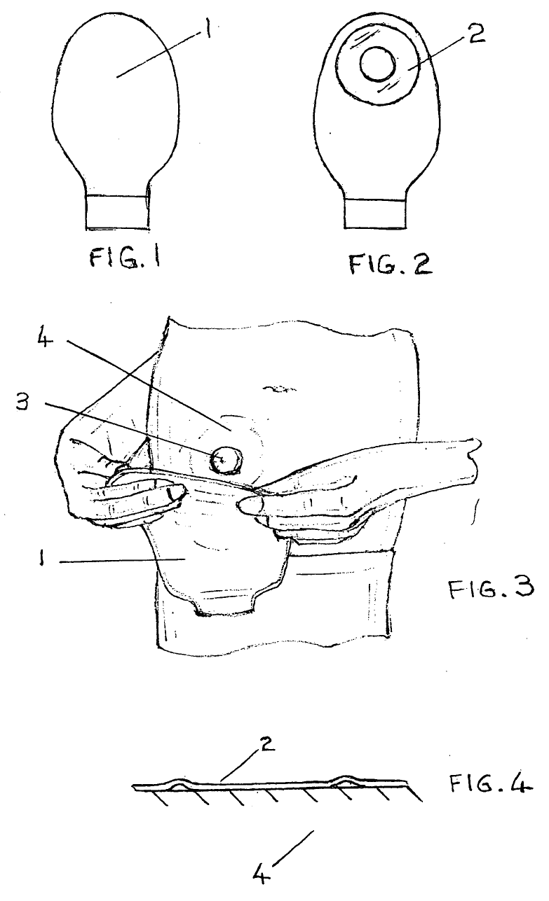

FIG. 2 is a rear view of a typical one-piece colostomy pouch showing the adhesive wafer 2.

FIG. 3 depicts a typical one-piece colostomy pouch 1, being applied to a stoma 3 and hernia 4 by the standard method.

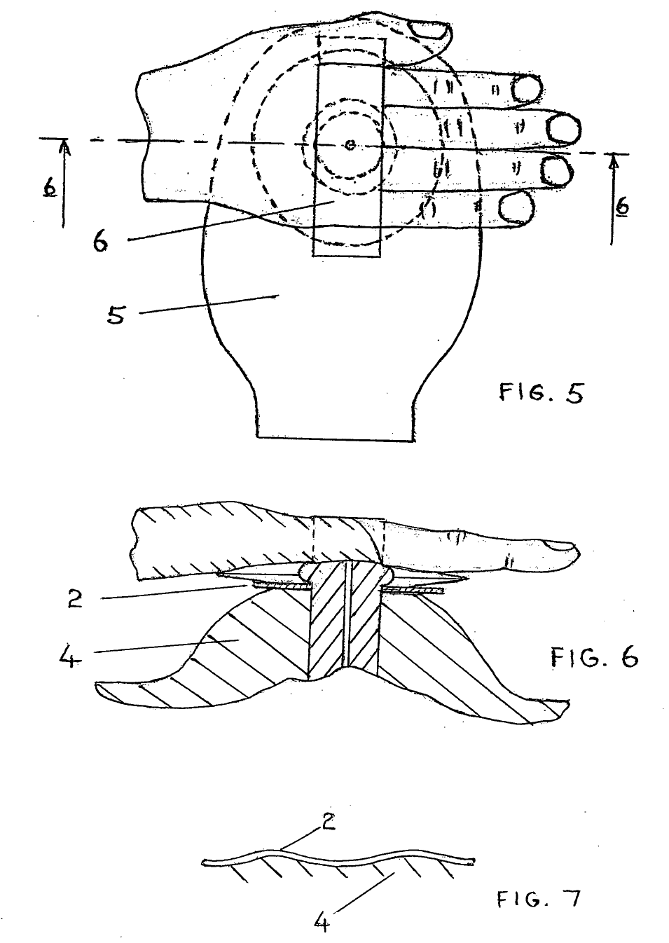

FIG. 4 is an edgewise representation of the wafer 2 of the standard pouch showing the wrinkles and potential leakage channels formed at the periphery following application by the standard procedure.

FIG. 5 is a front view of the improved pouch 5, embodying a handle 6, being applied by the improved procedure.

FIG. 6 is a sectional view of the improved pouch being applied by the improved procedure.

FIG. 7 is an edgewise representation of the wafer 2 of the improved pouch showing the wrinkles formed at the periphery following application by the improved procedure. The skin of the hernia 4 is seen to conform with the wrinkles which are wavelike with typically an amplitude of 2 mm and a wavelength of 30 mm.

I claim:

1. A one-piece colostomy pouch embodying a handle which provides a purchase for the open palm of one hand on the front of the pouch.

2. A handle according to claim 1 which facilitates the fixing of the pouch in place over the stoma by one simple push by the open palm of the hand, and leads automatically to a more effective seal than is obtained by the existing standard procedure.
